(19) United States
(12) Patent Application Publication
(10) Pub. No.: US 2008/0045817 A1
(43) Pub. Date: Feb. 21, 2008

(54) Apparatus and Method for Performing Othogonal Polarized Spectral Imaging (Opsi)

(75) Inventors: Michael Cornelis Van Beek, Eindhoven (NL); Egbert Lenderink, Waalre (NL); Robert Frans Maria Hendriks, Eindhoven (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS NV, Eindhoven (NL)

(21) Appl. No.: 10/596,562

(22) PCT Filed: Dec. 20, 2004
(86) PCT No.: PCT/IB04/52862

§ 371 Date: May 25, 2007

(30) Foreign Application Priority Data
Dec. 22, 2003 (EP) 03104918.2

(51) Int. Cl. A61B 5/00

(52) U.S. Cl. 600/310

(57) ABSTRACT

Provided is a method and an apparatus for detection of objects below the surface of diffuse scattering media, in particular blood capillaries in organs such as the skin of human beings, using Orthogonal Polarized Spectral Imaging (OPSI), according to the invention comprising the steps of: imaging the object in question at at least two different angles so as to obtain a shift of position in the imaging plane; and subsequently comparing relative shifts of objects in the two images so as to obtain coordinates of the imaged objects with respect to the organ surface.

The present invention relates to a method and system for detection of objects below the surface of diffuse scattering media, in particular blood capillaries in organs such as the skin of human beings, using Orthogonal Polarized Spectral Imaging (OPSI) as described by the preamble of independent patent claim 1.

It is a well know tendency in all areas of medical care to provide methods and systems that render possible a minimal or non-invasive treatment of patients inter alia to reduce risks as well as stress for the patient. In line with this tendency, projects have been set up to provide methods of non-invasive blood analysis. In Non-Invasive Blood Analysis, one of the possibilities is to measure the concentration of various analytes in blood in vivo by means of confocal Raman spectroscopy.

To achieve a Raman signal from blood instead of skin, the blood capillaries near the skin surface have to be visualized and the Raman detection volume has to be aimed at one of these capillaries. Blood capillaries close to the skin surface have a diameter of 5 to 15 μm. Confocal detection keeps the source of the collected Raman signal well confined in all three dimensions in a spot of <5×5×10 μm³. This makes it possible to collect a Raman signal from blood without a background signal from skin tissue if the focus is located in a blood capillary.

In this respect, a simple, cheap, and robust method of visualizing blood vessels close to the surface of organs is said Orthogonal Polarized Spectral Imaging (OPSI). Medical applications of Orthogonal Polarization Spectral Imaging can be taken, for example, from WO 01/22741, which is incorporated by reference herein. A recent test has shown that it is also possible to use OPSI to visualize blood capillaries in the human skin. In OPSI, polarized light is incident on the skin through a polarizing beam splitter. Part of the light reflects directly from the surface (specular reflection). Another part penetrates into the skin, where it scatters once or several times before it is absorbed or is re-emitted from the skin surface (diffuse reflection). In any of these scattering events there is a chance that the polarization of the incident light is changed. Light that is directly reflected or that penetrates only slightly into the skin will scatter only once or a few times before it is re-emitted and will mostly retain its initial polarization. On the other hand, light that penetrates more deeply into the skin undergoes multiple scattering and is depolarized before re-emitted back towards the surface. When looking at the subject through a second polarizer oriented precisely orthogonal to that of the first polarizer, light reflected from the surface or the upper parts of the skin is largely suppressed, whereas light that has penetrated deeper into the skin is mostly detected. As a result the image looks as if it were back-illuminated. Since wavelengths below 590 nm are strongly absorbed by blood, however, the blood vessels will appear dark in the OPSI image.

The reliability of a measured concentration of analytes in blood directly depends on the ability to direct the Raman detection volume inside a blood vessel. However, even though Orthogonal Polarized Spectral Imaging (OPSI) is a method of detecting blood capillaries in human skin, it is essentially a 2-dimensional technique, whereas a 3-dimensional image would be desirable to be able to aim the Raman detection volume exactly. While the lateral resolution of OPSI is of the same order of magnitude as the Raman technique, OPSI hardly provides any depth information. The only depth discrimination available is caused by the depth of focus of the imaging objective. As the capillary moves out of the focal plane, it is blurred. Using the sharpness of the imaged blood vessels to determine the depth of the vessels has several disadvantages: it is not very precise; when the capillary is seen as blurred it is not a priori clear whether the capillary is above or below the focal plane; when the capillary is seen as blurred it is not a priori clear what the distance is between the blood capillary and the focal plane. An additional complication is that even in-focus images of blood capillaries are blurred because of light scattering in the skin tissue above the blood capillary. The lack of reliable depth information makes it difficult to aim the Raman laser at a blood vessel.

Accordingly, it is an object of the present invention to provide a method and system for detection of objects below the surface of diffuse scattering media, in particular blood capillaries in organs such as the skin of human beings, using Orthogonal Polarized Spectral Imaging (OPSI), which provides a more precise localization of the objects, and in particular capillaries in the human skin.

This object is achieved by the features according to the independent claims, while the features as contained in the dependent claims describe preferred and useful embodiments.

Provided is a method for detection of objects below the surface of diffuse scattering media, in particular blood capillaries in organs such as the skin of human beings, using Orthogonal Polarized Spectral Imaging (OPSI), according to the invention comprising the steps of: imaging the object in question at at least two different angles so as to obtain a shift of position in the imaging plane; and subsequently comparing relative shifts of objects in the two images so as to obtain coordinates of the imaged objects with respect to the focal plane.

Accordingly, it is proposed to use a stereoscopic variety of OPSI to obtain depth information, wherein the blood capillaries are imaged at different angles, resulting in a shift of position in the image plane. It can be determined from the direction of the shift whether the blood capillary is above or below the focal plane, while the distance between the blood capillary and the focal plane can be calculated from the size of the shift. Stereoscopy is a well-known technique for conventional microscopy. The object is imaged at different angles, and depth information is obtained by comparing relative shifts of objects in the two images. The human brain does this automatically when the eyes separately view the two images. Image analysis algorithms are also able to extract this information and quantify it.

Modern stereomicroscopes are based on two different principles. In the so-called Greenough design, two identical objectives are used at different angles. In the so-called telescopic design or common objective design, two partial microscope systems are arranged in parallel with each other and use the same main objective. In a preferred embodiment of the invention, the angle between the light paths of the at least two images is chosen to be between 10 and 30 degrees.

Furthermore, non-invasive blood analysis by confocal Raman spectroscopy uses a relatively high magnification factor and high numerical aperture (NA) objective with a short working distance to focus the Raman laser and to collect the Raman signal. For ease of construction and alignment and because of space and cost limitations, it is advantageous to use the same objective for OPSI. There are basically two ways to obtain a stereoscopic image using a single objective: illuminating only a part of the objective with a parallel beam or illuminating the whole objective at a certain angle.

OPSI uses light with a wavelength of 540 to 580 mm for detecting blood vessels in human skin. A lateral resolution of 1 μm is preferable for OPSI imaging, which can be achieved by using a NA of 0.35. The relation between the depth resolution Δz and the stereoscopic angle α is given by

Tan α=0,5Δx/Δz.

Where Δx is the lateral resolution of the system. The factor 0.5 arises because imaging from the left (−α) and from the right (+α) are compared. Some typical values are given in the Table below:

| | Stereoscopic angle |
|---|---|
| Depth resolution (μm) | (degrees) |
| 1 | 27 |
| 2 | 14 |
| 5 | 6 |

For a NA=0.9 objective, the maximum angle at which light can travel in object space is 64°. For a lateral resolution of 1 μm, an effective NA of 0.35 an angle of 21° is required. Therefore, the maximum stereoscopic angle (neglecting other limitations like geometric constraints in image space) is 43°. The highest depth resolution of 0.54 μm is achieved at this angle.

Furthermore, an apparatus for stereoscopic Orthogonal Polarized Scattering Imaging (OPSI) is provided for imaging objects below the surface of diffuse scattering media, in particular blood capillaries in organs such as the skin of human beings, comprising at least a light source providing polarized light, an imaging device such as a CCD-camera, a beam splitter, which preferably is a polarized beam splitter, a focusing device such as an objective, or a mirror, and means for imaging the object at two different imaging angles, consecutively or at the same time. The light source is preferably arranged to illuminate a diffuse scattering medium, which upon this illumination illuminates the object with depolarized light. The means for imaging the object may be formed by two objectives having different imaging angles or by a single main objective, and a scanning mirror for shifting the imaging beam in its path from the polarizing beam splitter to the imaging device. The two imaging angles preferably differ by 10 to 30 degrees.

Furthermore, separate imaging devices may be provided for each image, or, as an alternative, a shutter for alternating transmission of one of the two images is provided, which is preferably located between the polarizing beam splitter and the imaging device and which may be embodied as a rotating-aperture shutter, a liquid crystal cell shutter, or any other suitable means. The imaging device may be for example, a CCD or CMOS camera.

The apparatus may further comprise a data processor for determining a position of the object, which position includes at least information about the z-axis parallel to the optical axis.

The apparatus may further comprise a spectroscopic analysis system having a spectroscopic light source which may be laser for providing a spectroscopic light beam, a spectroscopic light beam positioning device for directing the spectroscopic light beam to the object in dependence of the position of the object determined by the data processor. The spectroscopic analysis system may be identical to that described in WO 02/057759.

Further features and advantages of the present invention will become more apparent for those skilled in the art upon reading of the following description of preferred embodiments in connection with the annexed Figures, in which:

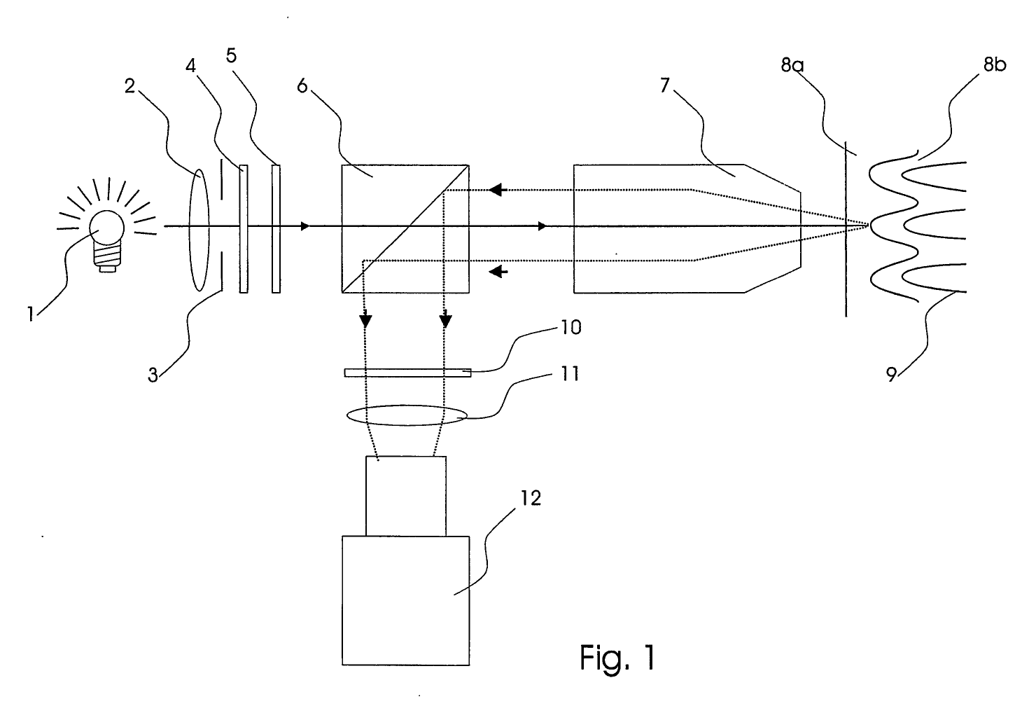

FIG. 1 is a schematic representation of a setup for OPSI;

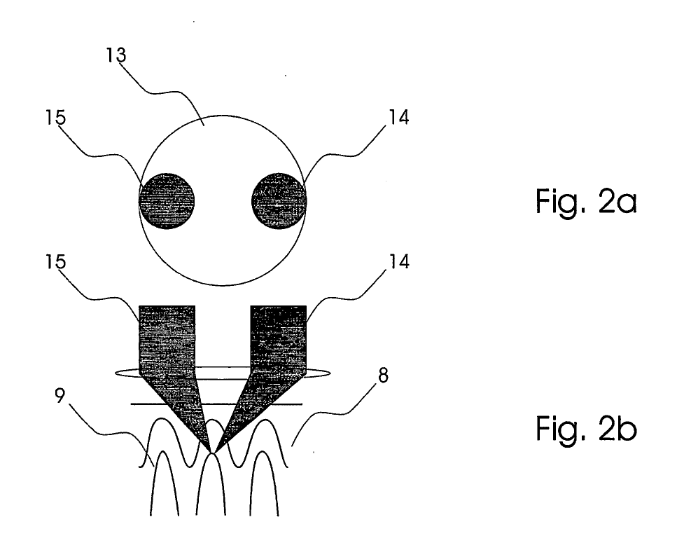

FIG. 2a is a schematic representation of the exit pupil of the imaging objective with OPSI light paths using parallel beams in plan view;

FIG. 2b is a side view of FIG. 2a;

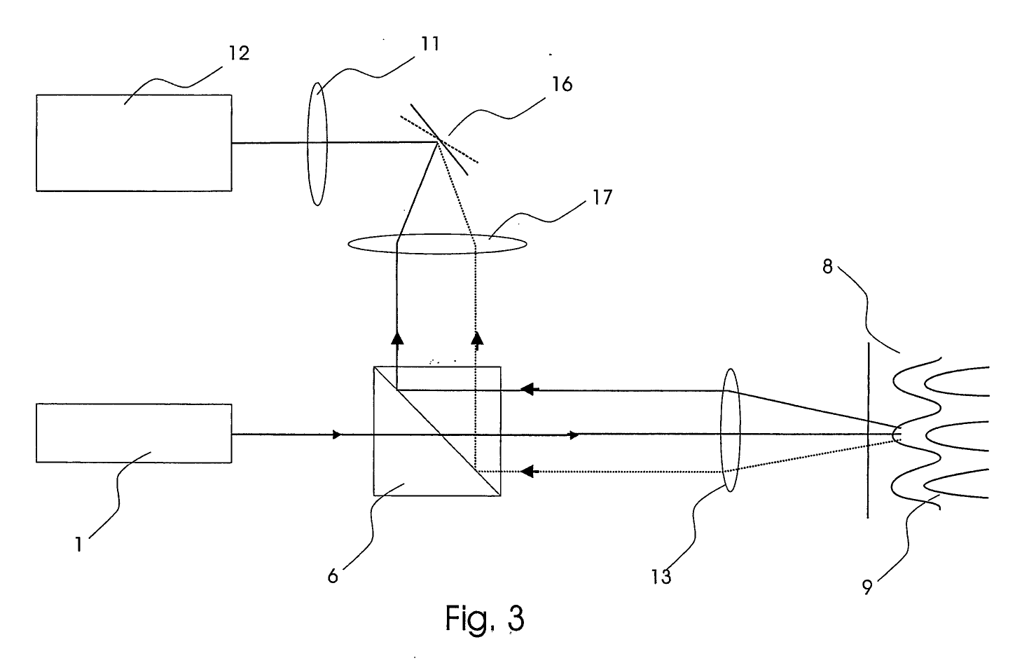

FIG. 3 shows an embodiment of the OPSI setup using parallel imaging beams;

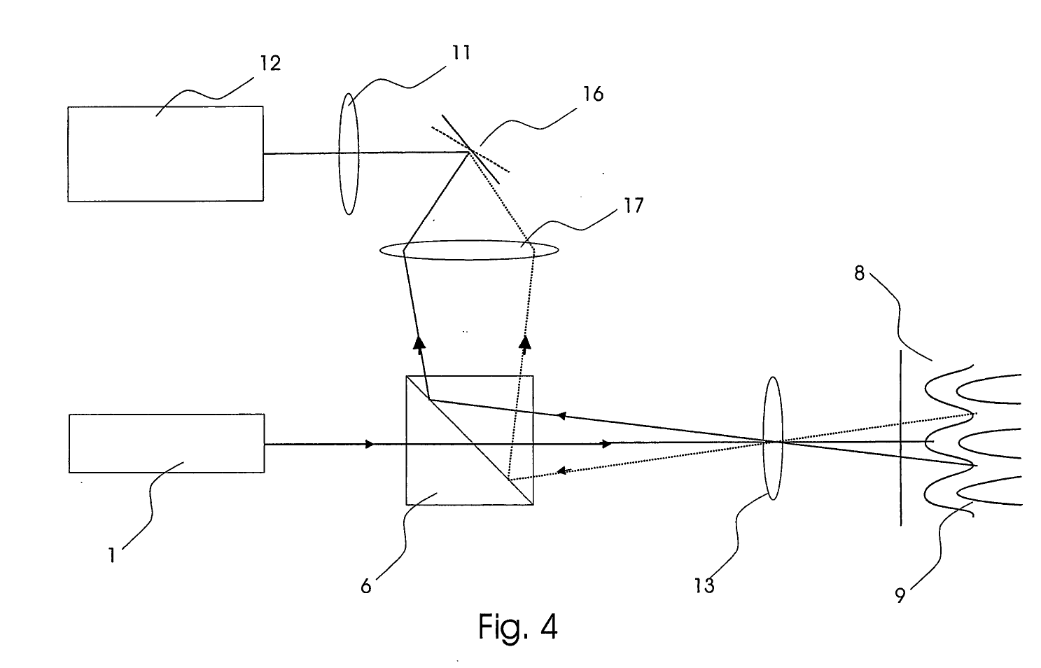

FIG. 4 shows an embodiment using the same objective and tilted imaging beams; and

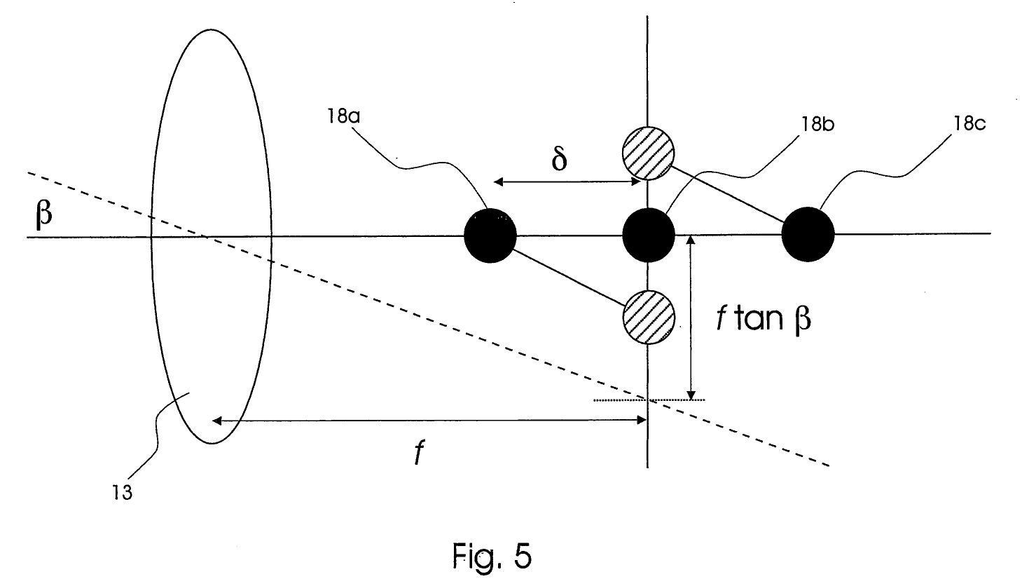

FIG. 5 shows the schematic position of blood vessels in an image as a function of the viewing angle and position relative to the focal plane.

FIG. 1 schematic ally shows a typical setup for OPSI, comprising a light source 1, such as a lamp, a laser, an LED, etc., a condenser 2, diaphragm 3, a color filter 4, a polarizer 5, a polarizing beam splitter 6, and an objective 7. Furthermore, FIG. 1 shows a skin 8 consisting of (a) epidermis, and (b) dermis, together with blood capillaries 9. Finally, an analyzer 10 is shown, wherein polarization is effected perpendicularly to polarizer 4, a lens 11, and a CCD camera 12.

FIG. 2a is a plan view of an exit pupil 13 of the imaging objective with OPSI light paths using parallel beams 14, 15. A non-invasive blood analyzer uses an objective with a NA of 0.9. A lateral resolution of 1 or 2 μm is required for OPSI imaging, which can be achieved by using an objective with a NA of 0.35. Since the NA required for OPSI (0.35) is much smaller than the NA available (0.9), it is possible to use only a fraction of the pupil 13 area for imaging. The different stereoscopic angles can be achieved by illuminating different areas of the pupil 13. Using parallel beams 14, 15, the blood vessels 9 in the focal plane are imaged in the same position if observed at the two stereoscopic angles. Vessels 9 that lie in front of or behind the focal plane have different positions in the two images. A possible embodiment is shown in FIG. 3.

The position of the imaging beam in the objective pupil 13 can be shifted by means of a scanning (rotating) mirror 16 and a relaying lens 17. If the distance between this lens 17 and the scanning mirror 16 equals the focal distance of the relaying lens 17, a tilt of the mirror 16 results in a parallel displacement of the imaging beam in the objective pupil 13. The distance between the objective pupil 13 and the blood vessel 9 is equal to the focal distance of the objective pupil 13 (corrected for the refractive index of human skin).

An alternative embodiment is shown in FIG. 4, where the same elements as in the previous Figures have been provided with corresponding reference signs. A polarizing beam splitter 6 separates the light paths of the illumination system and the imaging system. The imaging system contains a scanning mirror 16 and a relaying lens 17 such that the pivot point on the scanning mirror 16 is imaged on the center of the objective lens 13. An imaging lens is used to image the focal plane of the objective lens 13 onto a CCD camera.

As the scanning mirror 16 performs a wobbling motion, the OPSI image moves. Objects that are in front of or above the focal plane will move less than objects behind or below the focal plane. Objects that are in the focal plane will move over a distance Mf tan β, where M is the magnification factor of the OPSI system, f is the focal length of the objective, and β is the viewing angle through the microscope objective. β is related to the scanning angle σ of scanning mirror 16 as follows: tan β=(A/B ) tan 2 σ, where A is the distance from the scanning mirror 16 to the relaying lens 17 and B is the distance from the relaying lens 17 to the objective lens 13.

Objects that are at a distance delta above the focal plane will move over a distance that is slightly smaller, M (f−δ) tan β, whereas objects at a distance δ below the focal plane will move over a distance that is slightly greater, M (f+δ) tan β, cf. FIG. 5.

FIG. 5 shows the schematic positions of blood vessels 18, The three blood vessels 18a, 18b, 18c shown in FIG. 5 all overlap in the case of β=0, but their projections on the focal plane all have different displacements for β≠0.

Besides the embodiments described above, other embodiments are possible such as, for example, a single imaging device which includes a replacement for the scanning mirror by a rotating wedge or by two shifting wedges. It is also possible to use two imaging devices looking through the objective at different angles. This has the advantage that there are no moving parts and that the images from both sides can be detected simultaneously. The amount of de-focus can be determined from the obtained images by means of a correlation function or by subtracting the two images.

Provided is a method and an apparatus for detection of objects below the surface of diffuse scattering media, in particular blood capillaries in organs such as the skin of human beings, using Orthogonal Polarized Spectral Imaging (OPSI), according to the invention comprising the steps of: imaging the object in question at least two different angles so as to obtain a shift of position in the imaging plane; and subsequently comparing relative shifts of objects in the two images so as to obtain coordinates of the imaged objects with respect to the organ surface.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of other elements or steps than those listed in a claim. The word “a” or “an” preceding an element does not exclude the presence of a plurality of such elements.

1. An apparatus for performing Orthogonal Polarized Spectral Imaging for imaging objects below the surface of diffuse scattering media, comprising a light source for providing polarized light, an imaging device, a beam splitter, a focusing device, and means for imaging the object at two different imaging angles.

2. The apparatus according to claim 1, wherein the means for imaging the object is formed by two objectives having different imaging angles.

3. The apparatus according to claim 1, wherein the means for imaging the object is formed by a main objective, a scanning mirror, and at least one of a rotating wedge and two shifting wedges for shifting an imaging beam path from the polarizing beam splitter to the imaging device .

4. The apparatus according to claim 1, wherein a separate imaging device is provided for each image.

5. The apparatus according to claim 4, wherein a shutter is provided for transmitting each of the two images in alternation.

6. The apparatus according to claim 5, wherein the shutter is located between the polarizing beam splitter (6) and the imaging device.

7. The apparatus according to claim 5, wherein the shutter is one of a rotating-aperture shutter and a liquid crystal cell shutter.

8. (canceled)

9. The apparatus according to claim 1, wherein the two imaging angles differ by at least approximately 10 degrees.

10. Apparatus according to claim 1, wherein the imaging devices are one of CCD-cameras, CMOS-sensors, and a combination of CCD-cameras and CMOS-sensors.

11. (canceled)

12. The apparatus according to claim 1, characterized in further comprising a data processor for determining a position of the object, the position including at least information about the z-axis which is parallel to the optical axis.

13. The apparatus according to claim 12, further comprising a spectroscopic analysis system, with a spectroscopic light source and a spectroscopic light beam positioning device for directing the spectroscopic light beam to the object in dependence of the position of the object determined by the data processor.

14. A method for detection of objects below the surface of diffuse scattering media using Orthogonal Polarized Spectral Imaging, comprising the steps of: imaging the object in question at at least two different angles so as to obtain a shift of position in an imaging plane; and determining a shift in the object imaged at the two different angles so as to obtain coordinates of the imaged objects.

15. The method according to claim 14, wherein a direction of the shift determines whether the imaged object is above or below focal plane.

16. The method according to claim 14, wherein a distance between the object and a focal plane is calculated from the size of the shift.

17. The method according to claim 14, wherein the two imaging angles differ by at least approximately 10 degrees.

18. (canceled)

19. The method according to claim 18, wherein part of the objective (7) is illuminated with a parallel beam so as to obtain the at least two images.

20. The method according to claim 18, wherein the entire objective is illuminated at a defined angle so as to obtain the at least two images.

21. A blood analysis system comprising:
a light source for providing polarized light directed toward an object,
means for imaging the object at two different imaging angles to obtain a shift of position in an imaging plane; and
means for determining a shift of the object imaged at the two different angles so as to obtain coordinates of the imaged object.

22. The blood analysis system of claim 21, wherein a direction of the shift determines whether the imaged object is above or below a focal plane.

23. The blood analysis system of claim 21, wherein a distance between the object and a focal plane is calculated from the size of the shift.
